(19) United States
(12) Patent Application Publication
(10) Pub. No.: US 2018/0221333 A1
(43) Pub. Date: Aug. 9, 2018

(54) OPTIMIZED CANNABIS-BASED APHRODISIAC AND MOOD ENHANCER

(71) Applicant: RISE RESEARCH INC, Surrey (CA)

(72) Inventors: Chris W. Dollard, Surrey (CA); Anton R. Mattadeen, Toronto (CA)

(21) Appl. No.: 15/926,198

(22) Filed: Mar. 20, 2018

Related U.S. Application Data
(60) Provisional application No. 62/474,670, filed on Mar. 22, 2017.

(51) Int. Cl. A61K 31/352; A61K 36/185; A61K 31/05; A61K 36/534; A61K 9/00; A61K 36/068; A61K 31/015; A61K 31/01; A61K 31/045; A61P 15/00

(52) U.S. Cl. CPC A61K 31/352; A61K 36/185; A61K 31/05; A61K 36/534; A61P 15/00; A61K 36/068; A61K 31/015; A61K 31/01; A61K 31/045; A61K 9/0053

(57) ABSTRACT

A formulated cannabis product includes a cannabinoid mix and carrier processed by nano-emulsification. The cannabinoid mix in one embodiment includes between 0.1-0.3% Cannabinol (CBN). This cannabinoid mix is processed into a product yielding between 10-100 μg of CBN in each dose. The cannabinoid mix can include primary cannabinoids extracted from cannabis sativa (hemp or marijuana) plant material including tetrahydrocannabinol, cannabidiol, and combinations thereof. The cannabinoid mix includes isolated terpenes selected from the group consisting of, D-Limonene, α-Pinene, Myrcene, Linalool, and combinations thereof. These terpenes have a purity of 99% or greater. The isolated terpenes are from cannabis sativa plant material comprising between 0.2-10% of the cannabinoid mix. In another embodiment, at least a portion of these terpenes are derived from plants other than cannabis sativa. In a further embodiment, the formulated product includes a terpene rich essential oil derived from peppermint

### FIELD OF THE INVENTION

The present invention pertains to formulated products using cannabis sativa to achieve aphrodisiac and sexual enhancement effects.

### BACKGROUND OF THE INVENTION

Sildenafil is marketed under the trademark Viagra® and is a prescription medication used to treat sexual dysfunction in males. It has been widely successful because it operates to increase blood flow to the genitals in males. Sildenafil has not been widely shown to be useful in females.

Known side effects of sildenafil medication include dizziness, headache, flushing, or stomach upset may occur. Vision changes such as increased sensitivity to light, blurred vision, or trouble telling blue and green colors apart may also occur. Some users report that there are more serious side effects including joint pain and inflammation, bone loss, hearing loss, vision problems, and anemia. It is unclear whether these are direct results of long term use, or consequences that people with heart disease risk would experience without sildenafil.

US20050266108A1 to Flockhart et al, published 2015 Dec. 1 discloses a method of purifying and extracting cannabinoids using solvents.

US20170000744A1 to Kaufman, published 2017 Jan. 5 discloses a lipid nanoparticle composition carrier for cannabinoids. Kaufman encapsulated cannabinoids with phospholipid nanoparticles.

WO2016098112A1 to Sinai et al., published 2016 Jun. 23 discloses a condom comprising cannabis derived compositions for sexual enhancement and decrease of erectile dysfunction. Wearing the condom enables direct delivery of cannabinoids to the penile and vaginal locations.

U.S. Pat. No. 9,603,887 B2 to Brooks J. Kelly, published 28 Mar. 2017, discloses an enzyme inhibitor to improve efficacy of oral cannabinoid formulations, but does not discuss the use of such formulations for sexual enhancement including aphrodisiac effects.

What is desired is an aphrodisiac for both men and women. What is also desired is a way to alleviate the occurrence of erectile dysfunction in men, address low libido issues in women, and create an enhanced shared sexual experience for consenting partners. What is also desired is a medication to enhance sexual performance that has little, if any undesirable side effects. What is also desired is a medication that is enjoyable to consume orally.

### SUMMARY OF THE INVENTION

The invention is a formulation, process and delivery methodology that produces an aphrodisiac derived from a specific combination of cannabinoids and terpenoids present in cannabis sativa. The invention yields both a physiological and psychological effects, increasing the synchronization of mental, emotional and physical processes known to activate sexual arousal—whether that arousal is generated by natural means or by the use of a pharmaceutical Erectile Dysfunction (ED) solution in the case of men, or by use of a Female Sexual Disorder (FSD) solution in the case of women.

Cannabinoids are a class of diverse chemical compounds that act on cannabinoid receptors in mammalian cells. There are over a hundred known cannabinoids, many of which are found in cannabis sativa, as well as other plants. Plant derived cannabinoids are called phytocannabinoids. Some cannabinoids are synthesized and others are produced naturally in mammals. Those produced in the body of a mammal are called endocanabinoids. Cannabinoids operate in a number of ways that are being studied, and they often effect neurotransmitter function in vivo.

The invention includes a formulation and delivery method that enables a consumer to rapidly absorb particular cannabinoids and other constituents into the body. In one embodiment, the formulation uses a carrier selected from the group consisting of, water, silicone gel or combinations thereof to improve bioavailability of the active cannabinoid constituents of the formulation, and to hasten absorption.

While some cultivars of cannabis sativa plant material have traditionally developed a reputation for yielding aphrodisiac effects and sexual performance, the present invention identifies and formulates the key chemical constituents that create some of the pharmacological effects of pharmaceutical drugs, while using a herbal formulation.

The present invention includes unique modifications to the natural cannabinoid and terpenoid profiles of cannabis sativa, particularly the cannabinoid profiles that optimize the aphrodisiac effects. The present invention can also be used to create conditions that stimulate the desired increased psycho-physiological response while moderating any delimiting or negative side effects associated with tetrahydrocannabiniol (THC), which is found in abundance in many cannabis derived formulations. The main isomer of THC found in typical cannabis formulations is trans-Δ⁹-tetrahydrocannabinol. The main isomer of THC that is found in unprocessed plant material is Tetrahydrocannabinolic acid (THC-A).

The present invention is particularly suited to utilizing hemp as a cannabinoid source, although many plants produce cannabinoids. Hemp produces Cannabidiol (CBD) and Cannabidiolic Acid (CBD-A) in greater concentrations in the flower, leaves and other hemp biomass. These are the primary cannabinoids found in hemp. Interestingly, Cannabidiolic Acid naturally oxidizes to form CBD. This can occur under heat, exposure to ambient air, exposure to light including ultraviolet light. Products manufactured from hemp, therefore typically include CBD as an oxidative byproduct of the CBD-A, which is the most abundant cannabinoid in the living hemp plant.

THC-A and THC are also found in Hemp, but in small quantities measured in parts per million. THC-A also oxidizes. The oxidative byproduct of THC-A is THC. Typically less than 0.3% of the hemp flower is THC.

Cannabinol (CBN) is a less understood cannabinoid. It has some very interesting seditative effects. Although its concentration in hemp is typically under 100 ppm, and more likely less than 10 ppm, it does have noticeable effects for people even in small concentration. For example a dose of 10-20mg of THC is common in edible products. Oral dosage of Cannabinol (CBN) is typically in the microgram μg amounts. 10-100 μg of CBN may have a noticeable sedative effect in humans.

Although hemp products may contain small amounts of THC, and this is legal, the amounts of THC contained in hemp products do not produce noticeable psychoactive effects in humans due to the small concentrations.

Both CBD and CBN are not strictly regulated in most jurisdictions. Cannabinol (CBN) is formed as a metabolite of THC. Accordingly if the less than 0.3% of THC in a hemp product is converted to CBN, then the hemp product can have noticeable sedative, slight psychoactive, and mind expanding effects. This can enable a user to feel more connected emotionally, energetically and physically with another. Sensitivity to touch may be enhanced. Both males and females have reported that sexual contact is made natural and more fluid when a bioactive amount of CBN is consumed prior to sex.

Accordingly, the present invention includes processing a cannabinoid extract, or raw cannabinoid oil, to transform at least a portion of the THC into CBN.

In a preferred embodiment utilizing hemp oil, residual THC in the hemp oil is converted to CBN to maximize the effects of CBN. It can be appreciated that where 0.3% THC is found in a hemp extract, and this is converted to approximately 0.3% CBN, that this will have noticeable bioactive effects on a user. Preferably, this is accomplished by heating the hemp oil to within ten (10) degrees ° C. of the vaporization temperature of the THC.

The vaporization temperature of THC is 175° to 200° C. at 1 atm of pressure. If the hemp oil is held at above 165° C. for a proper amount of time, then a portion of the THC will convert into CBN.

The present invention includes a hemp-derived cannabinoid extract or mixture (cannabinoid mix) including between 0.01%-0.3% CBN. This enables a dosage of CBN of between 100-1000 μg. A ratio of between 1:50 to 1:30 CBN:CBD is enabled in a dose to enhance mood and sexual appetite, or simply to enable a sedative effect, or to improve sleep. Using a hemp-derived cannabinoid extract avoids regulatory hurdles posed by marijuana-derived extracts.

The invention enables the use of adult-use or medical cannabis products for health, recreational and other purposes including increasing libido, sexual function, and overall sexual enhancement. More specifically, the invention will help individuals or couples initiate and achieve an enhanced sexual experience characterized by shifting their mood and overall psycho-physiological state that could assist in improving sexual pleasure and acting as a component in the remediation of sexual dysfunction. The invention can be used to achieve desired results in both men and women.

The formulation of the present invention includes isolated cannabinoids including terpenoids found in cannabis sativa including but not limited to THC. In one embodiment, supplemental ingredients include horny goat weed, guinea hen weed, and combinations thereof.

A method of making the formulation includes pre-processing of raw plant material through curing cannabis sativa flower, leaf material, or combinations thereof Cannabinoids are extracted from the cured cannabis sativa material. Preferably the step of decarboxylation converts various cannabinoids into their non-acid form, i.e. conversion of THC-A to THC. Decarboxylation is accomplished by heating in one embodiment of the present invention.

In one embodiment the formulation includes selected extracted cannabinoid and other components of cannabis sativa. The selected extracted components are purified and isolated. Preferably, the cannabinoid components are extracted from cannabis material using CO₂ extraction, then separated using flash chromatography, which separates compounds based on polarity and hydrophobicity. This yields numerous cannabinoid fractions, including terpenes, which can be recombined later according to a formulation described herein. Additionally, terpenes and other components from other plant materials can supplement the formulation in accordance with the present invention.

In an alternate embodiment, some cannabinoids from cannabis sativa are isolated, and selected phytocannabinoids isolated from plant sources other than cannabis sativa are combined. In a variation of this embodiment, synthetic cannabinoids are combined in desired proportion to achieve the goals of the present invention.

The formulation delivered to the user consists of the following items on a per recommended dose basis.

The optimal cannabinoid mix is disclosed in terms of a single dose. Preferably a single dose includes 5 mg to 10 mg of delta 9 Tetrahydrocannabinol (THC—C₂₁H₃₀O₂) in decarboxylated (non-acid, activated) form. The cannabinoid mix includes at least 20%-50% THC of the cannabinoid mix on a weight to weight basis.

The cannabinoid mix and components thereof disclosed herein do not include dilution achieved by any carrier such as water or silicone gel.

While 10 Mg of THC is normally considered a minimal dose for oral delivery, it has been discovered that doses exceeding 10 mg may reduce the desired aphrodisiac effects of the present invention. With inexperienced users of cannabis, this undesired effect may begin to occur with a dose as small as 5 mg. Accordingly, the present invention requires any dosage include less than 10 mg of THC and preferably less than 5 mg of THC.

Further, the formulation of the present invention includes components including CBD that may have an antagonistic effect on CB1 and CB2 receptor agonists including THC. In this way excessive psychoactive effects of excessive THC, which can impair sexual desire, are reduced.

The cannabinoid mix includes Cannabidiol (CBD—C₂₁H₃₀O₂) in the range of 0.5% and 5.0% of the cannabinoid mix. In another embodiment 0.05-0.5 mg of CBD is included in a single dose. The use of CBD helps counter-balance the negative effects of THC such as sleepiness, memory impairment and potential for paranoia. It has also been shown to exhibit anti-inflammatory, anti-anxiety and anti-depression properties. CBD may moderate or inhibit the psychoactive effects of THC.

The cannabinoid mix includes Cannabigerol (CBG—C₂₁H₃₂O₂) in the range of 0.5% and 5.0% of the cannabinoid mix. In another embodiment 0.05-0.5 mg of CBG is in a single dose. CBG is a precursor chemical to THC, and exhibits numerous properties including modulating anxiety effects and reduction of intraocular pressure. Modulation of anxiety achieves relaxation. Relaxation better enables individuals to overcome resistance to intimate relations. Resistance to and issues surrounding intimacy may be a primary psychological reason for sexual dysfunction in many.

The cannabinoid mix includes D-Limonene in the range of 0.05% to 3.0% of the cannabinoid mix. In another embodiment 0.005-0.3 mg of D-Limonene is in a single dose. Many anecdotal reports concerning the use of cannabis sativa cultivars for aphrodisiac use all share a relatively high level of D-Limonene. This terpenoid's known effects include anti-stress, mood elevation, anti-fungal, anti-bacterial, anti-carcinogenic, anti-depression and acts as an aphrodisiac when combined with the components in this formulation in the concentrations expressed. D-limonene can be derived from cannabis sativa and numerous other plants

The cannabinoid mix includes α-Pinene in the range of 0.05% to 3.0% of the single dose. In another embodiment 0.005-0.3 mg of α-Pinene is in a single dose. One of the properties of α-Pinene is a mitigation of the common “brain fog” associated with THC use, during and after. In one embodiment 0.005-0.3 mg of a-Pinene is in a single dose. Common effects of α-Pinene include heightened mental clarity, memory retention, and attenuation of some effects of THC. It is found in cannabis sativa and pine tree resin, sage brush and camphorweed. A foggy feeling is a barrier to intimacy, and removing this barrier enables engagement of partners with intimacy before and during sexual relations.

The cannabinoid mix includes Myrcene in the range of 0.05% to 3.0% of the single dose. Myrcene is a monoterpene. In an embodiment 0.005-0.3 mg of Myrcene is in a single dose. It has a relaxing and sedative effect, which can be counterproductive in service of the invention if present in too high a ratio. Works synergistically with CBG and THC. Myrcene can be derived from bay leaves, ylang-ylang, wild thyme, parsley, hops and cannabis sativa.

Linalool in the range of 0.05% to 3.0% of the single dose. Linalool is a terpene alcohol known to reduce anxiety and produces a pleasant floral odor. In an embodiment 0.005-0.3 mg of Linalool is in a single dose. Linalool may help to improve water solubility of THC.

Peppermint oil in the range of 0.05% to 3.0% of the single dose. In another embodiment 0.005-0.3 mg of peppermint essential oil is in a single dose. Peppermint has a history of acting as an aphrodisiac by stimulating senses and altering mood, particularly in women. Peppermint's aroma performs a dual purpose by creating a dominant flavor that becomes part of the invention's user experience. Peppermint includes cytochrome P450 enzyme inhibitors for increasing the time of bio-activity of THC in vivo.

An emulsification process that prepares and mixes the formulated components i.e. the cannabinoids including terpenoids, and non-cannabinoid terpenoids, and other components such as the enabling surfactants in a water medium, or another medium that provides an efficient delivery format such as silicone gel.

Preferably, emulsification is achieved through nano-emulsion. Nano-emulsion reduces compounds in size, usually on the order of droplet sizes below approximately 300 nanometers (nm), to achieve improved water solubility, kinetic stability, higher potency, improved transdermal capability, and improved bio-availability. Nanoemulsions with droplet sizes smaller than 100 nm become optically translucent, with increasing clarity and stability as the droplet sizes diminish. The preferred specification for this invention is to attain droplet sizes smaller than 100 nm to provide translucence in the final product.

The emulsification process is carried out in combination with a substance called surfactants that assists in the breakup of the dispersed phase droplets. The formula ingredients are combined with surfactants, such as a type of oil, before the nanoemulsion process.

In one embodiment, a high-amplitude ultrasonic processor is utilized to deliver high ultrasonic amplitude on the order of 70-90 microns to asymmetrically implode vacuum bubbles within the formulation. The ultrasonic energy generates micron-sized cavitation jets that break apart components of the formulation into small enough sizes to enable a high degree of water solubility, and improved bioavailability.

In another embodiment, laser energy is utilized to generate cavitation jets in the formulation. Preferably, the cavitation jets are directed to uniformly and efficiently emulsify the formulation.

It can be appreciated at the use of a lipid, i.e. peppermint oil enables a liposome, or nano-sized liposome (nanosome) coating of the THC molecule, which improves bioavailability at the cellular level of the THC.

The formulation provides a pre-designed emulsion of cannabinoids and terpenoids, which are specific chemicals in specific proportion ranges to be effective for enhancement of sexual pleasure, stimulation, intensification and mood improvement. The terpenoid profile is designed to mitigate unwanted side effects of certain cannabinoids so that both inexperienced THC users and experienced THC users can enjoy the product.

An optional enhancement of the natural or optimized cannabinoid and terpenoid patterns through the addition of non-cannabis derived ingredients to create additional synergistic effects. These ingredients may be derived from botanical and/or chemical sources.

Pure cannabinoids such as delta 9 Tetrahydrocannabinol in acid form are crystalline and need to be decarboxylated through heat to activate certain properties. THC is only minimally water soluble under most circumstances.

Terpenoids are also referred to as terpenes. Terpenoids are lipids that are typically extracted as essential oils from plants and normally have low solubility in water. The present invention includes an emulsification process to create a water based formulation that is effective in yielding shelf stability, improved bioavailability, and optimal product consistency.

### METHOD EXAMPLE

A method of making the formulation of the present invention includes: providing raw cannabis flowers, drying the raw cannabis flowers, extracting oil from cannabis flowers using common techniques such as super-critical fluid extraction with CO₂ as a solvent, or a combination of CO₂ and other solvents. Refining the extracted oil to remove waxes and other residual plant matter using a process known as “winterization”. Next, the method includes separating the cannabinoid compounds of interest in a manner that renders compounds with a 99% or greater purity. Flash chromatography is preferably used to separate the cannabinoid compounds. The method includes heating the cannabinoids to achieve decarboxylation on the primary cannabinoids including THC and CBD. The method includes mixing cannabinoids, terpenoids and a carrier medium such as water to create a mixture. Emulsifying the mixture to created a formulated product. Preferably, the formulated product is emulsified using nano-emulsification to improve bioavailability of the cannabinoids.

The product is packaged in the form of an oral spray, or other orally consumable medium.

A unique feature of the formula and process is that the invention may replicate or enhance known cannabis sativa cultivar cannabinoid-terpenoid profiles. In one embodiment of the invention, the cannabis plant material is a strain that has a specific ratio range of compounds that generally and traditionally tends to provide aphrodisiac effects. The active compounds are identified and isolated. A product is made using this cannabinoid-terpenoid profile, with enhancement by addition or removal of selected bioactive terpenoids. One embodiment of the invention includes a cannabinoid mix that can be vaporized without the need for a carrier such as water.

The challenge in creating an orally consumed product, such as this invention describes, is that when manufacturing the product, the process of extraction and decarboxylation burns off or removes the majority of the terpenes, such that the user is generally just getting non-acid forms of the cannabinoids less the more volatile terpenes. By vaporizing or combusting (by smoking) the raw (but cured) plant material (or cold-extracted oil) does the user acquire the benefit of the terpenes at the moment of cannabinoid decarboxylation and terpene boiling points. However, not all people want to smoke or vaporize cannabis and prefer other delivery methods.

### Product

The present invention enables a decarboxilated THC or CBD product to deliver a desired terpene profile mimicking natural plant materials, or a desired terpene profile augmented by design.

In one embodiment, the invention mimics the cannabinoid-terpenoid profile of a particular cannabis sativa strain, except that the primary cannabinoids i.e. THC and CBD are decarboxylated. This is possible by the addition of specified fresh, externally sourced terpenes to match the overall effect of a particular cannabis strain, including the terpenoid profile. The terpenes are selectively chosen, re-introduced and adjusted to amplify the aphrodisiac potential of the invention and to mitigate any negative side-effects of THC.

The invention could also be used for mood elevation, as the cultivars explored contain chemical properties that carry the ability to stimulate positive mood shifts and heightened feelings of euphoria. Specific chemical component blends will be further explored to define the specific range, types and percentages of terpenoids that produce the optimum mood elevation formulation.

Optimally, the invention includes less than 10 mg THC in each dose, and preferably less than 5 mg of THC, to mitigate psychoactive effects and reduction of aphrodisiac effect.

### BRIEF DESCRIPTION OF THE DRAWINGS

FIG. 1 is a flow diagram of the process of the present invention.

FIG. 2 is a cannabinol (CBN) molecule.

FIG. 3 is a cannabidiol (CBD) molecule.

FIG. 4 is a tetrahydrocannabinol (THC) molecule.

FIG. 5 is a tetrahydrocannabinolic acid (THC-A) molecule.

### DETAILED DESCRIPTION

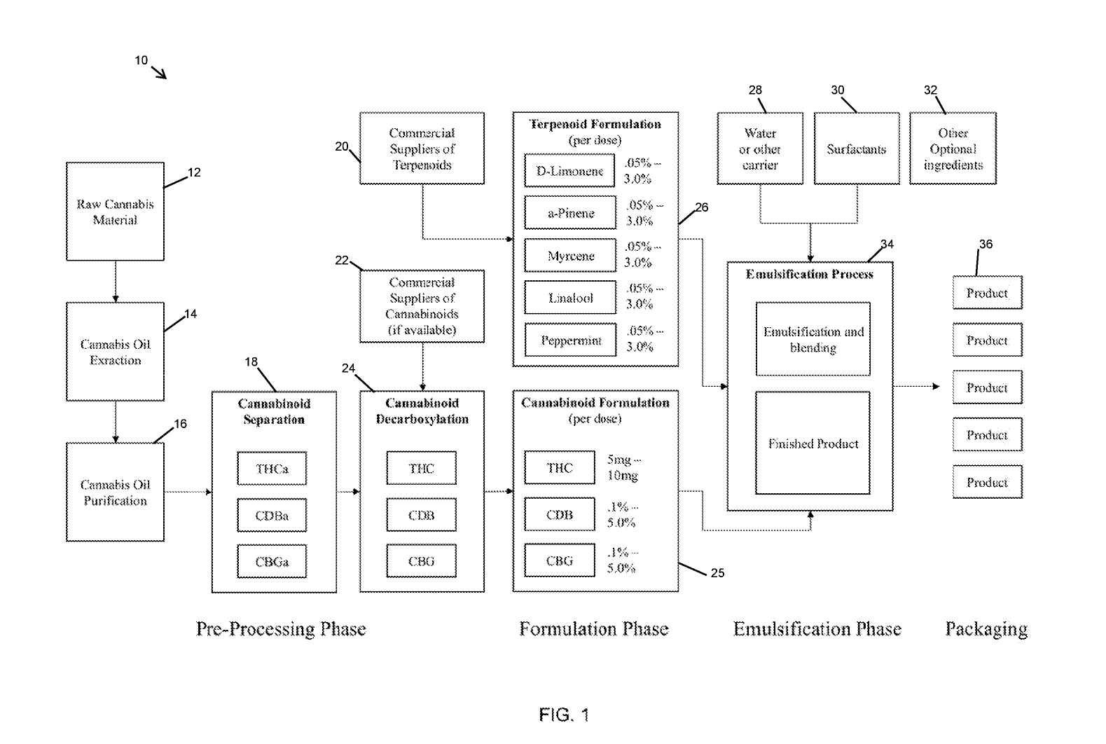

FIG. 1 shows a method in accordance with the present invention, generally designated with the reference numeral 10. The method 10 includes a first phase. The first phase includes the step 12 of providing raw cannabis material, the step 14 of extracting cannabis oil from the raw cannabis material, and the step of 16 of purifying the extracted cannabis oil.

Preferably the step 14 of extracting is accomplished without the use of toxic solvents. For example, the extraction methodology can include utilizing supercritical carbon dioxide. The step 16 of purification of cannabis oil can be accomplished through flash chromatography. While these methods are currently some of the more efficient and safe methods of extracting cannabinoids, it should be appreciated that numerous oil extraction and purification protocols can be developed and utilized in accordance with the present invention.

The method 10 includes a preprocessing phase having the step 18 of separating primary cannabinoids and secondary cannabinoids from the cannabis oil. The primary cannabinoids are THC, CBD, and CBG. Initially these cannabinoids are in their acid form. The next preprocessing step 24 is decarboxylation of the cannabinoids into their non-acid, decarboxilated form.

In one embodiment the separation step 18 separates THC from hemp oil having less than 0.3% THC. The decarboxylation step 24 additionally includes converting at least a portion of the THC to Cannabinol (CBN) with heat.

The step 22 provides isolated commercially viable primary cannabinoids mixed with the purified cannabis oil of steps 16, 18 and 24. The step 20 provides isolated terpenoids (terpenes), which can be mixed or blended with the isolated cannabinoids of step 22.

The method includes a formulation phase. In the formulation phase includes the step 26 of formulating terpenoids. The terpenoids can be isolated terpenoids from step 20 or whole plant mixtures of terpenoids of step 18. The terpenoids are blended or mixed into a cannabis formulation through the emulsification process 34 or other process with whole plant separated cannabinoids of step 18, or isolated cannabinoids from the step 22.

These primary cannabinoids of step 24 are preferably in their decarboxilated form. THC can be further processed into CBN.

The weight of THC in this formulation phase is preferably between 5 to 25 mg, representing one dose. A portion of the THC can be converted into CBN so that one dose has 0.5-2 mg of CBN.

In an alternate embodiment less than 5 mg of THC are used to mitigate the psychoactive effects normally associated with THC. In another embodiment the cannabinoids are derived from hemp in step 12, and yield a CBN content after conversion of less than 0.3%, or less than ling per dose. Having a product with the reduced THC level also virtually eliminates the possibility that an overabundance of THC could have adverse effects and actually inhibit sexual aptitude and arousal. Having a micro dose of CBN i.e. less than 1 mg, can yield pleasant sedative effects. Accordingly those having sexual dysfunction caused by stress and anxiety may be more able to engage in sex.

The formulation phase also includes assembling and mixing selected terpenoids in step 26. These terpenoids are expressed as a percentage of the cannabinoid mix. The cannabinoids mix includes both cannabinoids and terpenoids. Terpenoids can be from any plant source, preferably from cannabis sativa. Terpenoids are also commercially available.

The formulation phase further includes assembling and mixing selected cannabinoids. In one example, THC is in a 5-10 mg quantity per does. CBD is 1-5% per dose by weight of the cannabinoid mix or 1-5 mg per dose. CBG is 1-5% per dose of by weight of the cannabinoid mix or 1-5mg per dose. Further CBN is less than 1% of the cannabinoid by mix, or approximately 0.5-1 mg per dose.

In one formulation embodiment, the cannabinoids are hemp derived, and comprise 5-150 mg of CBD and 0.01-0.5% CBN derived from conversion of THC present in the hemp extracted cannabinoids.

The formulation is achieved through the step of emulsification in step 34. Step 34 mixes the cannabinoids and terpenoids. The terpenoids and the cannabinoids are mixed together with one or more surfactants in the step 30, water and other carriers in step 28, and other optional ingredients in step 32, in preparation for the nano-emulsion process.

The mixture is emulsified to create a finished product. Nano-emulsification is used to inhibit settling or precipitation of certain cannabinoids are terpenoids from the finished product. Nano-emulsification also increases shelf life. Nano-emulsification also enables ease in delivery when the finished product is directed through an oral spray.

Oral spray is one optimal in one embodiment of the invention because the nano-sized particles are readily absorbed by the upper digestive tract. This avoids first pass metabolism associated with other orally consumable products such as pills and capsules.

In one embodiment other optional ingredients are added to the emulsification. For example, peppermint oil is added in one embodiment. Peppermint oil includes various additional terpenoids. Peppermint oil further includes, ascorbyl palmitate, which is a fat-soluble form of vitamin C. It functions as a Cytochrome P450 enzyme inhibitor. Utilizing peppermint oil may reduce the effects of first pass metabolism associated with orally consumed products that reach the central digestive tract by inhibiting the metabolization of cannabinoids in the liver.

The step of extraction 14 may be achieved with a modular and mobile system for supercritical fluid extraction and concentration of active components of cannabis. The system operates by utilizing a CO2 source and compressing and heating the CO₂. CO₂ is forced through an extractor including plant material and the CO₂ carries oil from the plant material to an extract chamber with the separator. The CO₂ is separated from the extract and returned to the CO₂ source.

The extract is pumped into a purification module which purifies and isolates various cannabinoids from the oil. The extract is then formulated and emulsified utilizing nano-emulsification techniques. Such techniques may include high speed mixing, ultrasonic nano-emulsification, and laser light nano-emulsification. Ultrasound and laser light can be adapted to achieve a phenomenon known as capitation in liquids having an aqueous component. Cavitation is a boiling phenomenon that produces supersonic jets from vaporization of the aqueous component and the jets can split compounds into their molecular components achieving optimal nano-emulsification. Further, nano-emulsification can be achieved by chemical means.

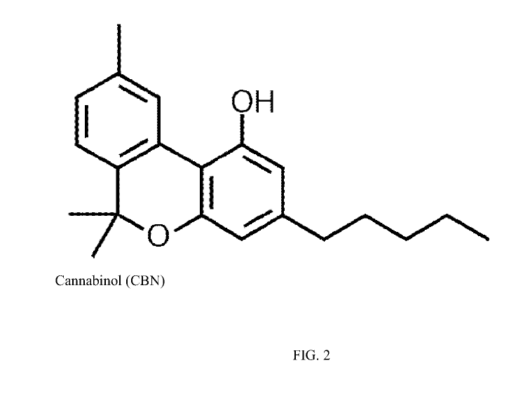

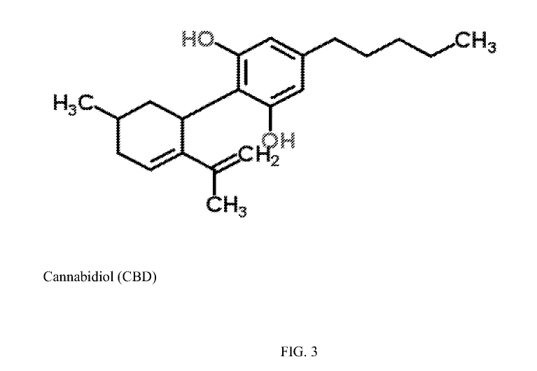

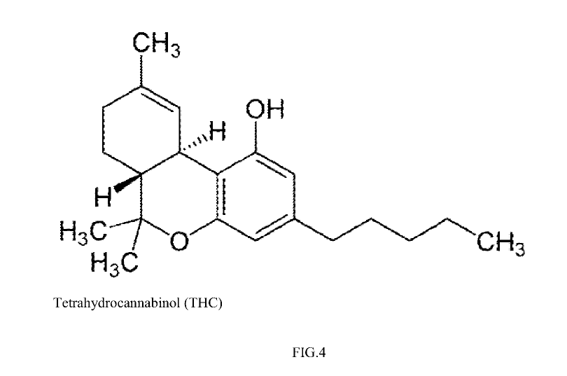

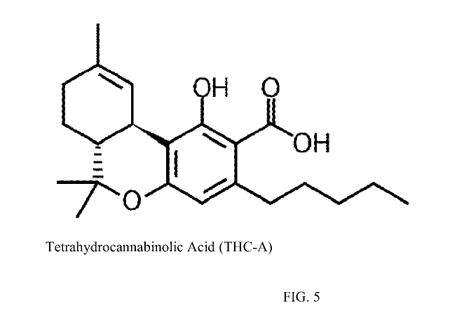

FIGS. 2-5 are various molecular diagrams. It can be appreciated that each molecule has various isoforms that can be substituted, supplemented or interchanged with those molecules shown herein.

While the present invention is described by way of example in this specification, the true scope and extend of the invention is specified in the appended claims. The invention is particularly adapted to orally consumable products including sprays, gel-caps, tinctures, and any other orally consumable form. Various embodiments of the invention include gels, creams, ointments, and other topically applied products. Pulmonary delivery via sprays, inhalers, vaporizers are also contemplated in accordance with the present invention.

We claim:

1. A formulated cannabis product able to enhance sexual function, comprising:
a cannabinoid mix and carrier processed by nano-emulsification;
cannabinoids extracted from cannabis sativa plant material without solvents and being selected from the group consisting of tetrahydrocannabinol, cannabidiol, and combinations thereof;
the cannabinoids comprising at least 50% of the cannabinoid mix;
isolated terpenes selected from the group consisting of, D-Limonene, α-Pinene, Myrcene, Linalool, and combinations thereof;
the isolated terpenes derived from cannabis sativa plant material comprising between 0.2 -10% of the cannabinoid mix by weight; and
a terpene rich essential oil derived from a non-cannabis source and including a cytochrome P450 enzyme inhibitor.

2. The formulated cannabis product as set forth in claim 1, wherein the cannabinoid mix includes 0.01-0.03% Cannabinol (CBN) by weight.

3. The formulated cannabis product as set forth in claim 1, wherein the product includes Cannabinol (CBN) and Cannabidiol in a ratio of between 1:30 to 1:50 by weight.

4. The formulated cannabis product as set forth in claim 1, wherein the cannabinoid mix is derived from hemp and the product includes Cannabinol (CBN) and Cannabidiol in a ratio of between 1:30 to 1:50 by weight.

5. The formulated cannabis product as set forth in claim 4, further comprising peppermint oil.

6. A formulated cannabis product for oral delivery, comprising:
a cannabinoid mix and carrier processed by nano-emulsification;
cannabinol (CBN) in a concentration of between 0.01-0.3% by weight of the cannabinoid mix;
isolated terpenes selected from the group consisting of, D-Limonene, α-Pinene, Myrcene, Linalool, and combinations thereof,
the isolated terpenes derived from cannabis sativa plant material comprising between 0.2 -10% of the cannabinoid mix; and
a terpene rich essential oil derived from a non-cannabis source and including a cytochrome P450 enzyme inhibitor.

7. The formulated cannabis product as set forth in claim 6, wherein the product includes Cannabinol (CBN) and Cannabidiol (CBD) in a ratio of between 1:30 to 1:50 by weight.

8. The formulated cannabis product as set forth in claim 6, wherein the cannabinoid mix is derived solely from hemp and the product includes Cannabinol (CBN) and Cannabidiol in a ratio of between 1:30 to 1:50 by weight.

9. The formulated cannabis product as set forth in claim 8, further comprising peppermint oil.

10. A formulated cannabis product for oral delivery, comprising:
a cannabinoid mix and carrier processed by nano-emulsification;
cannabinol (CBN) in a concentration of between 0.01-0.3% by weight of the cannabinoid mix;
isolated terpenes selected from the group consisting of, D-Limonene, α-Pinene, Myrcene, Linalool, and combinations thereof,
the isolated terpenes derived from cannabis sativa plant material comprising between 0.2-10% of the cannabinoid mix; and
cordyceps sinensis mushroom.

11. The formulated cannabis product as set forth in claim 10, wherein the product includes Cannabinol (CBN) and Cannabidiol (CBD) in a ratio of between 1:30 to 1:50 by weight.

12. The formulated cannabis product as set forth in claim 10, wherein the cannabinoid mix is derived solely from hemp and the product includes Cannabinol (CBN) and Cannabidiol in a ratio of between 1:30 to 1:50 by weight.

13. The formulated cannabis product as set forth in claim 12, further comprising peppermint oil.

14. The formulated cannabis product as set forth in claim 12, wherein the cordyceps sinensis mushroom comprises a mushroom extract including a blend of both water and alcohol based extracted cordyceps mushroom.

15. The formulated cannabis product as set forth in claim 12, wherein the cordyceps sinensis mushroom is dried and powdered.
